SEQUENCE LISTING (32 sequences)

SEQ ID NO: 1 - DNA, 25 bp, Haemophilus influenzae
gttatctcgaaaattaacccagttg

SEQ ID NO: 2 - DNA, 25 bp, Haemophilus influenzae
cgatgaaaatggtcagccagttgaa

SEQ ID NO: 3 - DNA, 23 bp, Streptococcus pyogenes
gtcgtttcacgtattgtaccagt

SEQ ID NO: 4 - DNA, 23 bp, Streptococcus pyogenes
ttccagacggaacaccagttgac

SEQ ID NO: 5 - DNA, 22 bp, Streptococcus pneumoniae
ttccagacggaactccagtcga

SEQ ID NO: 6 - DNA, 22 bp, Streptococcus pneumoniae
cagacggaactccagtcgacat

SEQ ID NO: 7 - DNA, 21 bp, Pseudomonas aeruginosa
caacggcaccccggtcgacat

SEQ ID NO: 8 - DNA, 20 bp, Pseudomonas aeruginosa
tggaagacatgccgcacgat

SEQ ID NO: 9 - DNA, 21 bp, Legionella pneumophila
gcctgttgaggatatgccaca

SEQ ID NO: 10 - DNA, 24 bp, Legionella pneumophila
tggaagatggaacagcagtagaca

SEQ ID NO: 11 - DNA, 21 bp, Escherichia coli
tacgatgaaaacggtactccg

SEQ ID NO: 12 - DNA, 21 bp, Escherichia coli
caacccgatcgaagatatgcc

SEQ ID NO: 13 - DNA, 23 bp, Staphylococcus aureus
tatgccttacttaccagatggac

SEQ ID NO: 14 - DNA, 20 bp, Staphylococcus aureus
taccagatggacgtccgatc

SEQ ID NO: 15 - DNA, 19 bp, Mycoplasma pneumoniae
cagtagcggacatgcccca

SEQ ID NO: 16 - DNA, 25 bp, Mycoplasma pneumoniae
ttagaagatggtactccagtcgaca

SEQ ID NO: 17 - DNA, 21 bp, Neisseria gonorrhoeae
atggcggacggccgtcctgtg

SEQ ID NO: 18 - DNA, 26 bp, Neisseria gonorrhoeae
aaatggtaatcctgtagatatcgtac

SEQ ID NO: 19 - DNA, 22 bp, Corynebacterium diphtheriae
ctgcctcaggaagatatgccat

SEQ ID NO: 20 - DNA, 23 bp, Artificial Sequence
  primer
  misc_feature (3)..(3): y is c or t
  misc_feature (6)..(6): n is a or g or c or t
  misc_feature (9)..(9): h is a or c or t
  misc_feature (12)..(12): y is c or t
  misc_feature (15)..(15): w is a or t
  misc_feature (18)..(18): y is c or t
  misc_feature (21)..(21): r is a or g
gcyggncghcayggwaayaargg

SEQ ID NO: 21 - DNA, 19 bp, Artificial Sequence
  primer
  misc_feature (3)..(3): y is c or t
  misc_feature (6)..(6): s is c or g
  misc_feature (9)..(9): v is a or c or g
  misc_feature: d is a or g or t
  misc_feature (18)..(18): y is c or t
ggyacsccvagdgggttya

SEQ ID NO: 22 - DNA, 71 bp, Moraxella catarrhalis
ggttgtatcacgcatcatgccagttgaggatatgccatatgatgaaaatggtaatcctgtagatatcgtac

SEQ ID NO: 23 - DNA, 71 bp, Moraxella cuniculi
ggttgtatcacgcattatgccagttgaggatatgccttatgatgaaaacggcaatcctgtggacatcgtgc

SEQ ID NO: 24 - DNA, 71 bp, Moraxella caviae
cgtggtatcacgcatcatgccagtagaagacatgccttatgatgaaaatgkcaaccctgtggacatcgtgc

SEQ ID NO: 25 - DNA, 71 bp, Neisseria gonorrhoeae
tgtggtatctcgcattctgcctgtggaagacatgccgtacatggcggacggccgtcctgtggacatcgtac

SEQ ID NO: 26 - DNA, 71 bp, Haemophilus ducreyi
cgtcatctcgaagatcctgccgctcgaggacatgccgttcctggcggacggcaccccggtggacatcgtgc

SEQ ID NO: 27 - DNA, 71 bp, Haemophilus parainfluenzae
tgttatctcaaaaatcaaccctgtggaagatatgccatacgatgaaaacggtcaaccggttgaaatcgtat

SEQ ID NO: 28 - DNA, 71 bp, Streptococcus oralis
ggttgtctctcgtatcgttcctgtagaagacatgccttaccttccagatggaactccagtcgatatcatgt

SEQ ID NO: 29 - DNA, 71 bp, Streptococcus mitis
ggttgtctctcgtatcgttcctgtagaagatatgccttaccttccagatggaactccagtcgatatcatgt

SEQ ID NO: 30 - DNA, 71 bp, Corynebacterium diphtheriae
tgtcgtgggcaagatcctgcctcaggaagatatgccattcatgccagacggcaccccagtggacatcatcc

SEQ ID NO: 31 - DNA, 71 bp, Legionella pneumophila
ggtgatctcgattgttgtgcctgttgaggatatgccacatatggaagatggaacagcagtagacatcgttc

SEQ ID NO: 32 - DNA, 71 bp, Pasteurella pneumotropica
ggttatctcaaaaatcaatccggtggaagatatgccgtatgatgaaaacggtcaaccggttgaaattgtgt

(19) United States
(12) Patent Application Publication
(10) Pub. No.: US 2007/0243530 A1
(43) Pub. Date: Oct. 18, 2007

(54) Nucleic Acid Probes and Broad-Range Primers from Regions in Dna Directed Rna Polymerase Subunit B Genes, and Methods in Which They are Used

(75) Inventors: Jaan Palgi, Helsinki (FI); Jari Jalava, Turku (FI); Simo Nikkari, Turku (FI)

Correspondence Address: BUCHANAN, INGERSOLL & ROONEY PC, POST OFFICE BOX 1404, ALEXANDRIA, VA, 22313-1404, US

(73) Assignee: Mobidiag Oy, Helsinki (FI)

(21) Appl. No.: 10/583,329

(22) PCT Filed: Dec. 17, 2004
(86) PCT No.: PCT/FI04/00776

§ 371 Date: Jun. 16, 2006

(30) Foreign Application Priority Data
Dec. 19, 2003 (FI) 20031867

(51) Int. Cl. C12Q 1/68; C07H 21/04

(52) U.S. Cl. 435/6; 536/23.1; 536/24.33

(57) ABSTRACT

The invention relates to nucleic acid probes and to broad-range primers that are useful in the identification of bacterial species and the diagnosis of bacterial infections. Especially, the invention relates to specific nucleic acid probes that originate from hyper-variable regions situated near the conserved sequences of the gene for RNA polymerase beta subunit, rpoB (DNA directed RNA polymerase subunit B) of infection-causing bacteria. The invention also relates to broad-range primers originating from the conserved regions of rpoB genes. In addition, the invention relates to the use of these nucleic acid probes and broad-range primers in the diagnosis of bacterial infections as well as to diagnostic methods in which these nucleic acid probes and broad-range primers are used.

### FIELD OF THE INVENTION

The invention relates to nucleic acid probes and to broad-range primers that are useful in the identification of bacterial species and in the diagnosis of bacterial infections. Especially, the invention relates to specific nucleic acid probes that originate from hyper-variable regions situated near the conserved sequences of the gene region encoding for RNA polymerase beta subunit, rpoB (DNA directed RNA polymerase subunit B) of infection-causing bacteria. The invention also relates to broad-range primers originating from the conserved regions of rpoB genes. In addition, the invention relates to the use of these nucleic acid probes and broad-range primers in the diagnosis of bacterial infections as well as to diagnostic methods in which these nucleic acid probes and broad-range primers are used.

### BACKGROUND OF THE INVENTION

Respiratory tract infections are a common cause for physician office visits in Finland and worldwide. In addition to viruses, respiratory tract infections are caused by a variety of bacterial species. Streptococcus pyogenes (group A streptococcus) is an important causative agent of tonsillitis. The risk of severe complications, such as peritonsillar abscesses, is connected to untreated tonsillitis. Furthermore, sequelae of tonsillitis caused by S. pyogenes include rheumatic fever and glomerulonephritis that are severe, even fatal diseases. Besides viruses, the most important causative agents of pneumonia in outpatients are Streptococcus pneumoniae (pneumococcus), Mycoplasma pneumoniae, and Chiamydia pneumoniae from which pneumococcus is the most common and serious pathogen causing pneumonia. A less frequent pneumonia-causing bacterial species is Legionella pneumophila. Mycobacterium tuberculosis, on the other hand, causes pulmonary tuberculosis. In addition to pneumococcus, causative agents of maxillary sinusitis and inflammation of the middle ear (otitis media) include Haemophilus influenzae and Moraxella catarrhalis.

At the moment the diagnosis of bacterial respiratory tract infections is mainly based on bacterial culture testing. Bacterial culture testing is, however, relatively slow, and diagnostic methods based on cultivation usually give results only days, sometimes up to weeks after sampling. Furthermore, cultivation of bacteria is not always successful under laboratory conditions. This can be a consequence of either the fact that the culture method used is not applicable for the bacterial species in question or the fact that the patient has received antimicrobial therapy before the sample is taken. In the diagnosis of pharyngitis, rapid methods based on the antigen detection are good supplementary methods to bacterial culture testing, but they work only with a limited number of pathogens (group A streptococcus). In the diagnostics of some bacterial infections (e.g., C. pneumoniae and M. pneumoniae) serological methods can also be used, but these methods give results only after days or several weeks from the onset of infection. Thus, they are not necessarily helpful in the acute treatment of a patient.

Molecular methods based on amplification and hybridization of nucleic acids attempt to solve the above-mentioned problems of bacterial culture testing. With the help of these methods the pathogen is simultaneously detected and identified, resulting in more rapid diagnostics and obviating the need for time-consuming additional culture tests. Also, antibiotics do not interfere with molecular methods to such a degree as with culture testing.

One molecular method used in bacterial diagnostics is the so-called broad-range PCR (polymerase chain reaction) that is based on the use of broad-range primers. At the moment the most common broad-range PCR methods are based on the use of primers that recognize conserved DNA sequences of bacterial chromosomal genes encoding ribosomal RNA (16S rDNA/rRNA or 23S rDNA/rRNA). In bacterial identification based on broad-range PCR the actual identification step is carried out by sequencing the amplified PCR product. (See e.g., EP-B1 613 502, U.S. Pat. No. 6,001,564, and U.S. patent application Ser. No. 00/20,055,101). The direct identification of multibacterial infections requires sequencing of transformant libraries produced by cloning.

The broad-range bacterial PCR method has to some extent been applied to clinical bacterial diagnostics, although it is best suited for the identification of bacterial and fungal species from culture isolates, and for this purpose commercial tests, such as MicroSeq (Applied Biosystems), have also been developed. However, these tests are not widely used, because sequencing of the PCR product is time-consuming and labor-intensive, and the assays themselves and the necessary equipment, such as sequencing instruments, are expensive, and performing the tests requires specially trained personnel.

Another method used to some extent in bacterial diagnostics is classical specific oligonucleotide based PCR, or its application, the multiplex PCR method. In this method a mixture of bacterial species-specific primers is used. Hendolin et al. (Journal of Clinical Microbiology, 35: 11, 2854-2858, 1997) used the multiplex PCR method when identifying pathogens causing otitis media. In this method the broad-range primer originating from the conserved gene region of 16S rRNA was used as one PCR primer and the mixture of primers that consists of four different bacterial species-specific primers was used as another PCR primer. Species-specific primers were designed so that the amplified PCR product differs in length depending on from which bacterial species it is originating. The identification of pathogens is thus based on the length of the amplified PCR-fragment. Although the multiplex PCR procedure is relatively sensitive and rapid, it has some disadvantages. It is known that shorter DNA fragments are amplified more efficiently than longer DNA fragments. Therefore, if the same specimen includes two different bacterial species, the bacterial DNA of the shorter fragment is likely to be amplified more efficiently, which affects the sensitivity of the method. Furthermore, with multiplex PCR it is possible to identify only a few bacterial species simultaneously, because in practice it is impossible to design for example over a dozen specific primer pairs so that they would be functional under the same PCR conditions. Therefore, multiplex PCR is not applicable, e.g., to the diagnostics of respiratory tract infections in which more than ten clinically important pathogens need to be determined at the same time.

Bacteria of so-called normal flora can also cause problems in microbial diagnostics based on multiplex PCR. The genome of only a few dozen bacterial species has been wholly mapped and most of these bacterial species are known disease causative agents. Thus, there is very little information about the bacteria of normal flora and their DNA sequences. This is why the design of species-specific PCR primers and bacterial diagnostics based solely on PCR amplification are almost impossible.

The use of ribosomal RNA also includes some drawbacks. Distinguishing related bacterial species from each other with the help of rRNA molecules is difficult, because the sequences of these molecules do not contain enough variable regions when they are compared with each other. And even if differences between various bacterial species may be found, these variable sites are generally divided across the whole rRNA molecule (e.g., the length of 16S rRNA is about 1500 nucleotides), which limits the use of molecular methods for diagnostics. Thus in practice, the related bacterial species can only be distinguished from each other by sequencing the whole rRNA encoding gene. However, even this approach is not always sufficient to distinguish bacterial species from each other.

### BRIEF DESCRIPTION OF THE INVENTION

The purpose of the present invention is to provide tools and means, which are useful in bacterial diagnostics of infectious diseases, especially those causing respiratory tract infections and ear, nose and throat diseases, but which lack the drawbacks of bacterial diagnostics described above. In particular, the purpose of the invention is to provide novel tools and methods, which are useful in bacterial diagnostics based on molecular methods, the tools and methods being sensitive, effective, and species-specific, and being capable of identifying specifically only the desired bacterial species. A further purpose of the invention is to provide methods, by which it is possible to diagnose infectious bacteria substantially faster than previously possible, whereby correct and effective antimicrobial therapy can be prescribed to the patient at an earlier stage of the infection so that the duration of the infection becomes shorter and the risk of potentially harmful, even life-threatening complications is reduced.

The present invention provides bacterial species-specific oligonucleotide probes that originate from hyper-variable regions situated near the conserved regions of genes encoding DNA directed RNA polymerase [EC:2.7.7.6] subunit B, rpoB, these hyper-variable regions differing in the base sequences significantly in various bacterial species. With these bacterial species-specific probes the genome of infection-causing bacteria can be simultaneously detected and identified.

The invention also provides broad-range primers that originate from the conserved regions of rpoB genes encoding DNA directed RNA polymerase [EC:2.7.7.6] subunit B, and that efficiently amplify DNA of infection-causing bacteria even from clinical specimens, which include large amounts of foreign (non-bacterial) DNA.

Furthermore, the present invention provides simple, rapid, sensitive, and specific methods that overcome the drawbacks of the prior art. With these methods clinically important bacterial species can be reliably identified and diagnosed from clinical specimens or bacterial cultures.

The present invention relates to oligonucleotide probe sequences that hybridize under normal hybridization conditions with sequences of hyper-variable regions situated near the conserved sequences of rpoB genes encoding DNA directed RNA polymerase [EC:2.7.7.6] subunit B of bacteria that cause infections, especially infections of the respiratory tract and ear, nose and throat diseases, and comprise any one of the sequences identified by SEQ. ID. NR: 1 to 19, and/or reverse and/or complementary sequences thereof, or a functional fragment thereof.

Examples of bacterial species that cause infections, especially infections of the respiratory tract and ear, nose and throat diseases, include Haemophilus influenzae, Streptococcus pneumoniae, Streptococcus pyogenes, Pseudomonas aeruginosa, Staphylococcus aureus, Legionella pneumophila, Corynebacterium diphteriae, Mycoplasma pneumoniae, Escherichia coli, Moraxella catarrhalis and Neisseria gonorrhoeae. Specific examples include Streptococcus pneumoniae, Haemophilus influenzae, Staphylococcus aureus, and Corynebacterium diphteriae.

Preferably the length of oligonucleotide probe sequence of the invention is 15-30 nucleic acids, more preferably 19 to 30 and most preferably 19 to 26 nucleic acids.

The present invention also relates to the use of the above-mentioned oligonucleotide probe sequences in the detection, identification, or classification of bacterial species.

The present invention further relates to a mixture of oligonucleotide probes, which comprises any combination, and preferably all the sequences identified by SEQ. ID. NR: 1 to 19, and/or their reverse and/or complementary sequences, and/or functional fragments of the afore-mentioned sequences. In one preferred embodiment, the desired mixture of probes has been attached to a solid support. Preferably, an oligonucleotide probe mixture that comprises all sequences identified by SEQ. ID. NR. 1 to 19, and/or their reverse and/or complementary sequences, has been attached onto a solid support.

The present invention further relates to a novel mixture of DNA primers that comprises sequences that hybridize with the sequences of conserved regions of rpoB genes encoding DNA directed RNA polymerase [EC:2.7.7.6] subunit B of bacteria causing infections, and which comprise sequences identified by SEQ. ID. NR: 20 and 21 and/or their complementary sequences or functional fragments thereof.

The present invention also relates to the use of the above-mentioned mixture of primers in the amplification of rpoB.

Furthermore, the present invention relates to a diagnostic method for detecting and identifying bacteria causing infections in a clinical specimen, which comprises

a) amplifying DNA isolated from the clinical specimen using the a mixture of primers that comprises sequences that hybridize with the sequences of conserved regions of rpoB genes encoding DNA directed RNA polymerase [EC:2.7.7.6] subunit B of bacteria causing infections, and which comprise sequences identified by SEQ. ID. NR: 20 and 21 and/or their complementary sequences or functional fragments thereof.

b) contacting the amplified DNA with a desired combination of the oligonucleotide probe sequences that hybridize under normal hybridization conditions with sequences of hyper-variable regions situated near the conserved sequences of rpoB genes encoding DNA directed RNA polymerase [EC:2.7.7.6] subunit B of bacteria causing infections, and that are bacterial species-specific, under hybridization conditions, and

c) detecting the formation of a possible hybridization complex.

One preferred embodiment of the method of the invention comprises amplifying the DNA, isolated from the clinical specimen, by polymerase chain reaction, and contacting the amplified DNA with the bacterial species-specific oligonucleotide probes attached to the solid support.

In one preferred embodiment of the method of the invention, a suitably labeled nucleotide is used in the amplification of DNA isolated from the clinical specimen in order to generate a detectable target strand.

In another preferred embodiment of the method of the invention, the amplified and possibly labeled target DNA is contacted with a solid support, preferably with treated glass on which all the species-specific oligonucleotide probes of the invention having sequences identified by SEQ. ID. NR: 1 to 19 and/or their reverse and/or complementary sequences have been attached.

In a further preferred embodiment of the method of the invention, the amplified and possibly labeled target DNA is contacted with a solid support, preferably with treated glass, on which the specific oligonucleotide probes of the invention of one specified bacterium or a few specified bacteria causing respiratory tract infections have been attached, said specific oligonucleotide probes having corresponding sequences identified by SEQ. ID. NR. 1 and 2, 3 and 4, 5 and 6, 7 and 8, 9 and 10, 11 and 12, 13 and 14, 15 and 16, 17 and 18 or 19 from Table 3 and/or their reverse and/or complementary sequences thereof.

The present invention further relates to a diagnostic kit for use in the diagnosis of infection-causing bacteria, especially those causing respiratory tract infections, comprising

a) a DNA primer mixture of the invention as defined above,

b) a mixture of bacterial species-specific oligonucleotide probe sequences, optionally attached on a solid support, of the invention as defined above,

c) positive and optionally negative control probe sequences, and optionally

d) reagents required in the amplification, hybridisation, purification washing, and/or detection steps.

### BRIEF DESCRIPTION OF FIGURES

FIG. 1 shows an example of a hyper-variable rpoB gene region that is limited by the conserved sequences (Mycoplasma pneumoniae). The conserved regions bold typed and underlined, and they act as the annealing sites of rpoB primers. The hyper-variable region is between these conserved sequences and has been marked with small letters.

FIG. 2 shows an agarose gel electrophoresis analysis of the labeling-PCR result of the culture isolate bacteria. In the Figure: lane 1 is M. catarrhalis, lane 2 is M. cuniculi, lane 3 is M. caviae, lane 4 is N. gonorrhoeae, lane 5 is H. influenzae, lane 6 is H. ducreyi, lane 7 is H. parainfluenzae, lane 8 is S. pyogenes, lane 9 is S. pneumoniae, lane 10 is S. oralis, lane 11 is S. mitis, lane 12 is P. aeruginosa, lane 13 is C. diphtheriae, lane 14 is L. pneumophila, lane 15 is E. coli, lane 16 is P. pneumotropica, lane 17 is S. aureus, lane 18 is M. pneumoniae and M is a 100 base pair marker.

FIG. 3 shows an example of the results of hybridization on the microarray slide. The amplified DNA of the rpoB gene isolated from culture isolates of Streptococcus pneumoniae (pathogen) and Streptococcus oralis (normal flora), which belongs to the same genus, was used as the target strand to be hybridized (asymmetric Cy-5-dCTP labeled PCR product). The example slide the arrow points out the oligonucleotide spots, which bind the labeled target strand of S. pneumoniae. The oligonucleotide sequences in these spots are S. pneumoniae oligonucleotide probes 5 and 6 as shown in Table 3. Also a positive control oligonucleotide (broad-range PCR primer SEQ. ID. NR. 21) gave a signal. S. oralis-specific oligonucleotide spots are not detected on the glass slide.

### DETAILED DESCRIPTION OF THE INVENTION

The present invention is based on studies, which attempted to find more specific alternatives for the use of ribosomal RNA in the diagnostics of infection-causing bacteria. The study was focused on other genes that are vital or bacteria. The rpoB gene region encodes the subunit β of the DNA directed RNA polymerase [EC:2.7.7.6] that consists of three subunits α, β and β′ (rpoA, rpoB and rpoC, respectively). Function of the DNA directed RNA polymerase in bacteria is to catalyze the transcription of DNA.

Certain regions in proteins and, correspondingly, in genes have remained almost unchanged, i.e. conserved, during evolution. In this context the terms “a conserved region” or “conserved regions” refer to a region or regions of rpoB genes or proteins, whose nucleotide sequence or equivalently the amino acid sequence has remained nearly unchanged between different bacterial species causing various infections. Usually these conserved regions are the most important regions for the functioning of the protein. rpoB-molecules are, however, not as conserved as ribosomal RNA molecules. Because the molecules in question are proteins, the genes encoding these proteins include more differences at the nucleic acid level, due to the nature of their genetic code, than the genes encoding structural RNA molecules (e.g., 16S rRNA molecules). Additionally, rpoB molecules as a whole have not remained as unchanged as the structural RNA molecules during the evolution: short DNA fragments can be found in rpoB molecules where the differences between various bacterial species (including closely related bacterial species) are so great that these DNA sequences can be considered as species-specific. These sequences are hyper-variable sequences, which in this context refer to DNA sequences of the rpoB genes which differ in the nucleotide base sequence between different bacteria to an extent affording bacterial species-specificity and which are situated near the conserved sequences of the rpoB genes and optionally limited or marked out by the conserved sequences.

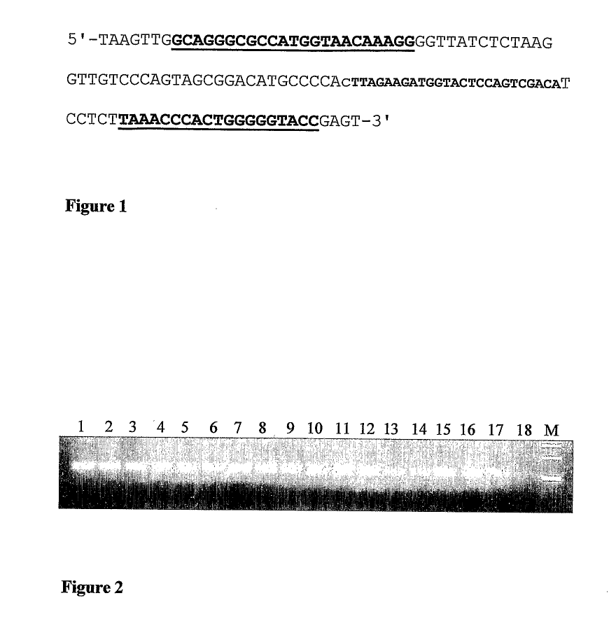

The above-mentioned features were utilized in the design of species-specific oligonucleotides for bacterial strains. In the design of species-specific probes (oligonucleotides) (Table 3) a planning strategy based on alignment was used. The rpoB genes of target bacterial species were aligned with the corresponding genes of the reference bacteria. The sequences were obtained from the EMBL public sequence database or, in the cases where such sequences were not available in public databases, they were produced by cloning the rpoB gene sequence fragments from the bacterial species. The sequences were produced by amplifying the desired rpoB DNA sequence fragment from bacterial culture isolates. The desired DNA sequence fragment was then cloned and sequenced. An example of the hyper-variable rpoB gene region of bacterial species Mycoplasma pneumoniae is shown in FIG. 1. Conserved regions are bold typed and underlined, and they act as the annealing sites of broad-range rpoB primers. The hyper-variable region, to which species-specific probes have been designed, is between these conserved sequences (smaller capital letters).

Alignment of the sequences was performed with the BioEdit program and the ClustalW alignment algorithm. The consensus sequence of the alignments was calculated and the suitably conserved regions were identified manually. These regions refer to sequence fragments that are conserved in the genes of the target bacterial species but are not found, at least entirely, in the genes of the reference bacterial species. Oligonucleotide sequences with the suitable length (e.g., 19-26 nucleotides) were selected from these areas for comparison analyses. The selected oligonucleotide sequences were compared to the EMBL prokaryotic sequence database using the FastA algorithm program. The oligonucleotide sequences having at least two mismatches when compared to rpoB sequences of non-target bacterial species were chosen for further analyses. Melting temperatures (Tm) of oligonucleotides were calculated and the formation of secondary structures (hairpin structures) was examined. The oligonucleotides without strong secondary structures and with Tm higher than 45° C. were selected for specificity testing. The specificity of the oligonucleotide probes was tested in laboratory conditions both with pure DNA samples isolated from various bacterial species (Table 2) and with clinical patient samples (Table 4).

The oligonucleotide probes of the present invention comprise the sequences identified by SEQ. ID. NR: 1 to 19, and/or their reverse and/or complementary sequences. They can be of different length, and only the desired species-specificity and functionality of these oligonucleotide probes in hybridization reactions determine their suitable length. The length of the oligonucleotide probes is generally 15 to 30, preferably 19 to 30 and most preferably 19 to 26 nucleotides. Furthermore, the probes can be modified in different ways (e.g., modified nucleotides, such as inosine, can be included). Also, various chemical compounds or groups (e.g. amino groups) or other molecules, such as labels necessary for the detection, can be attached to the probes, or they can be entirely unmodified. The sequences of the preferred bacterial species-specific probes and their specificities are presented in Table 3, and they have sequences identified by SEQ. ID. NR: 1 to 19. Naturally, reverse and complementary sequences of these oligonucleotide sequences are equally useful and preferable, as is obvious to a person skilled in the art. Similarly, functional fragments of the previously mentioned oligonucleotide sequences are useful as probes provided that species-specificity remains unchanged.

For designing the PCR primers, amino acid sequences of rpoB proteins of Chlamydia pneumoniae, Mycoplasma pneumoniae, Haemophilus influenzae, Streptococcus pneumoniae and Streptococcus pyogenes were aligned with the BioEdit program using the ClustalW alignment algorithm. In alignment studies many conserved regions were found and taken as the starting point for the broad-range primer design. The conserved amino acid sequences were reverse-translated to the corresponding nucleic acid sequences. Depending on the nature of the genetic code there were several degenerated sites in the primer sequences. On the basis of the conserved sequences several primer pairs were designed and tested in laboratory (specificity and sensitivity testing).

The aim of the present study was to find out a primer pair, which would on one hand amplify DNA from clinical specimens, and on the other hand also retain a high specificity so that rpoB proteins of all bacteria causing respiratory tract infections could be amplified. A functional primer pair is presented in Table 1. With this primer pair, all rpoB genes of bacteria that are phylogenetically distant from each other (Table 2) can be amplified even in the presence of large amounts of human DNA.

TABLE 1

rpoB broad-range primers

| | |
|---|---|
| Name of | SEQ. |
| the | ID. |

| | | |
|---|---|---|
| primer | Sequence 5′→3′ | NR |

| | | |
|---|---|---|
| rpoB2-for | GCYGGNCGHCAYGGWAAYAARGG | 20 |
| RPOb2-rew | GGYACSCCVAGDGGGTTYA | 21 |

In the primer sequences

D represents base A or G or T,

Y represents base C or T,

N represents base A or G or C or T,

H represents base A or C or T,

W represents base A or T,

R represents base A or G,

S represents base C or G and

V represents base A or C or G.

The primer mixtures, which consist of several different primer alternatives, are thus concerned. For example, in the case of primer rpoB2—for the mixture includes primers, in which W represents A (adenine) and primers, in which W represents T (thymine).

According to the invention, specific probes can be used for identification of infection-causing pathogens, especially bacteria that cause respiratory tract infections and ear, nose and throat diseases, in any suitable method, by which hybridization can be demonstrated. These methods are well known among the persons skilled in the art and they can be performed both in a solution and on a solid support that binds DNA, such as on nitrocellulose or nylon membrane, or on glass.

In one preferred embodiment of the method of the invention, bacterial identification is performed using the DNA microarray technology. In this context, a DNA microarray or a DNA chip refers to a small substrate on which known nucleic acid sequences have been attached in a predetermined order. If the nucleic acid fragments attached to the microarray are shorter than 100 base pairs (generally around 20-30 base pairs), the microarray is called an oligonucleotide array.

In the method of the present invention the sample to be analyzed can be a bacterial culture, a tissue fragment, a secretion sample, such as a sputum or brush sample, a blood sample, or another suitable sample, obtained from a patient suspected of suffering of an infectious disease. Especially the sample to be analyzed is a secretion sample suitable for clinical diagnostic applications.

DNA is isolated from the sample to be analyzed with any conventional method, such as with commercial DNA isolation kits (e.g., High Pure PCR Template Preparation Kit, Roche; NucleoSpin, BD Biosciences Clontech; or QlAamp DNA Mini-kit, Qiagen) or with conventional extraction with phenol-chloroform or equivalent organic solvents, either manually or with special devices that are suitable for performing DNA isolation. Commercial kits are preferably used because of their general availability, rapidity, and repeatability.

In the method of the present invention the reagents used in DNA amplification can be any reagents that are conventionally used for the amplification of DNA, and are well known among the persons skilled in the art. Suitable and cost-effective reagents, which are commercially available, include different types of Taq DNA polymerases and buffers therefor (e.g., Ampli-TaqGOLD, AmpliTaqLD, DyNAzyme, TaqPlus Precision, and HotStartTaq), nucleotides or pre-prepared mixtures of nucleotides (e.g., Sigma, Applied Biosystems, Amersham Biosystems), MgCl₂ (whereby a product from the manufacturer of the Taq polymerase is generally used), and Cy5-dCTP (e.g., NEN LifeSciences, Amersham Biosciences).

In the method of the present invention cloning can be performed with any conventionally known method, for example by using commercially available cloning kits (e.g., Qiagen PCR Cloning Kit, QIAGEN or TOPO TA Cloning Kit, Invitrogen). Sequencing of the cloning products can be performed with any sequencer suitable for this purpose (e.g., Applied Biosystems, model 373A, 377, or 3100, or Bio-Rad Sequi-Gen GT), or the products can be sequenced manually. The sequences can be analyzed manually or with sequence analysis programs designed for this purpose (e.g., Applied Biosystems Sequencer or Vector NTI Suite Version 7, InforMax).

The equipment used for amplification can also be any suitable device (e.g., TI Thermocycler, Biometra, or GenAmp PCR system 2700, Applied Biosystems). Practically all devices and equipment suitable for DNA amplification can be used, and amplification can also be performed manually by transferring reaction tubes from one temperature to another. In addition, amplification can be performed directly on a DNA microarray.

Purification of the PCR product can be performed with any commercial method (e.g., High Pure PCR Product Purification Kit, Roche; MicroSpin S-400, or S-300 HR Columns, Amersham Biosciences; or QlAquick PCR-purification-Kit, Qiagen) or using extraction with an organic solvent. The amplification product can also be used for the hybridization reaction as such without any further purification or extraction steps.

In order to form a single-stranded target strand any known digestion method can be used. These methods include, e.g., asymmetric PCR, exonuclease treatment, or the synthesis of a single-stranded target strand directly onto the microarray surface (e.g., matrixarray, Roche Applied Science). The invention also comprises applications in which a double-stranded PCR product can be used in the hybridization reaction. In the context of the present invention asymmetric PCR is the preferred method to generate a single-stranded target strand.

In the method of the present invention any suitable label can be used in order to produce a labeled target strand. Suitable labels include fluorescent labels (e.g., Cy5, Cy3, Cy2, TexasRed, FITC, Alexa 488, TMR, FluorX, ROX, TET, HEX), radioactive labels (e.g., ³²P, ³³P, ³³S), and chemiluminescent labels (e.g., HiLight Single-Color Kit). In the present invention the Cy5-dCTP fluorescent label (Amersham Biosciences) is preferred. The invention also comprises the applications in which no label is needed, such as those in which the detection of nucleic acids is based on electric impulse (e.g., the Motorola eSensor).

When hybridization takes place on a solid support, the probes used in hybridization can be attached onto the surface of the solid support by covalent or non-covalent binding. Alternatively, other chemical, electrochemical or equivalent attachment methods can be used. The substrate or support, on which the probes are attached, can be manufactured from glass, plastic, metal, nylon, nitrocellulose, polyacrylamide, silicon, or a combination of these materials, and the size of the substrate can vary from a couple of millimeters to a few centimeters. The surface of the substrate used can be treated with aminosilane or any other suitable surface treatment, such as epoxysilane, or alternatively a substrate that does not require any separate surface treatment can be used. A preferred substrate for the oligonucleotide probes is a microscopic glass slide treated with aminosilane (e.g., Genorama, Asper Biotech Ltd., Estonia).

The probes can be printed onto the surface of the microarray support with any commercially available arrayer that is suitable for this purpose (e.g., Qarray-mini arraying system, Lucidea Array Spotter, OmniGrid, or GeneMachines arrayer), or they can be pipetted manually onto the surface. Alternatively, the probes can be synthesized directly onto the surface by using photolithography.

The hybridization mixture used in hybridization can be different in its composition than what has been presented later in the working Examples, for example, the salt composition and/or the concentration can vary (e.g., 2-4×SSC or SSPE), or commercially available hybridization solutions can be used (e.g., ArrayHyb, Sigma). In addition, denaturing or stabilizing additives (e.g., formamide, DMSO, i.e. dimethyl sulfoxide) or substances that decrease non-specific binding (e.g., BSA, i.e. bovine serum albumin, or ssDNA, i.e. salmon sperm DNA) can be used in the hybridization mixture. Hybridization can be carried out in various hybridization temperatures (generally between 40-70° C.), and the time needed to perform hybridization can vary, depending on the application, from a few minutes to one day. Instead of a water bath, hybridization can be carried out, e.g., in an incubator or in a special hybridization device (e.g., GeneTAC HybStation or Lucidea Slidepro Hybridizer). The post-hybridization washing steps can vary in their duration, volume, temperature, and in the composition of the washing solution, and can therefore differ from the exemplified method. The washing steps of microarray slides can also be performed with a separate device. In some cases a washing step is not necessary, because the microarray slide can be analyzed immediately after hybridization. In a preferred method a +57° C. water bath is a suitable hybridization condition. The glass slides were hybridized for 14-16 hours in these conditions.

Microarrays or chips can be analyzed with any equipment or reader applicable for this purpose (e.g., GeneTAC UC4, GenePix Personal 4100A, or Agilent DNA Microarray Scanner). If the target strand has been marked with a fluorescent label, analysis can also be performed, e.g., with a fluorescent microscope. If a radioactive label has been used, the chip or membrane can be analyzed with autoradiography. If hybridization has been carried out on the surface of an electronic microarray and the analysis is thus based on electronic detection, the microarray can be analyzed with special equipment designed for this purpose.

The method of the present invention does not suffer from the problems of the prior art. The amplification step of the method of the present invention is very sensitive and a certain gene region rpoB from phylogenetically different bacterial species were amplified efficiently with broad-range primers of the invention regardless of whether the bacterial species were gram-negative or gram-positive (Table 2). Furthermore, the PCR product is short (about 114 base pairs), which improves the effectiveness of the amplification reaction.

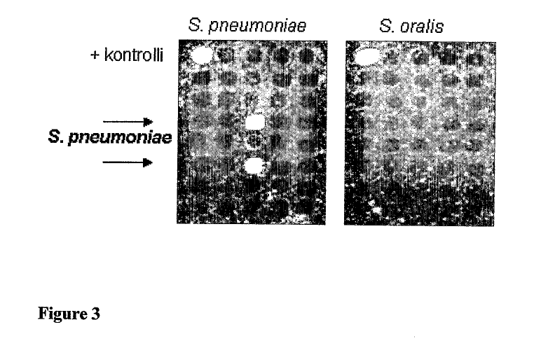

The bacterial species-specific probes have been designed for the rpoB gene region that is considerably more variable than for example the 16S rRNA gene region, which has previously been used in bacterial diagnostics. Although bacterial species of normal flora are also amplified efficiently with broad-range primers used in the present method, no false positive reactions occur, because the probes of the invention are very bacterial species-specific and identify only those bacteria for which they have been designed. When hybridizing the target strand amplified from culture isolates of Streptococcus pneumoniae onto a glass slide, the target strand will hybridize only with probes specific for Streptococcus pneumoniae and with positive controls. On the other hand, when hybridizing the target strand amplified from bacteria of normal flora, e.g. Streptococcus oralis, the product will not attach to any pathogen probe; in this case only positive control probes will emit a signal (FIG. 3). All specific oligonucleotide probes of the present invention were cross-tested with various bacterial species, including many bacterial species that belong to normal flora, and no cross-reactions were found to take place. Thus, the method of the present invention is considerably more sensitive and specific than the previously described methods of similar type.

An additional advantage of the present invention is the versatility it provides for the diagnosis of bacterial species causing infections. Methods and test kits can be assembled, as desired, for the analysis of a clinical specimen for the identification of a large number of bacterial pathogens, i.e. the screening of the clinical specimen for the disease-causing bacteria. Alternatively, methods and test kits can be designed for the specific identification of any individual bacterial pathogen(s). For instance, a special method and test kit can be designed for the detection of the diphtheria-causing pathogen, C. diphtheriae, whose rapid detection is utterly important for the correct treatment. Culture diagnosis of C. diphtheriae requires the use of special culture media that is not usually available in routine clinical diagnostic laboratories.

In the following, the present invention is more precisely illustrated with the Examples. When describing the method, references have been made to different equipment, materials, temperatures, chemicals, or equivalents used in this application. These can naturally be varied in a suitable way in different applications of the invention. Therefore, the present invention and its embodiments are not limited to the Examples described below.

### EXAMPLE 1

### The Design of PCR Primers according to the Invention

For the design of PCR primers, amino acid sequences of rpoB proteins of Chlamydia pneumoniae, Mycoplasma pneumoniae, Haemophilus influenzae, Streptococcus pneumoniae and Streptococcus pyogenes were aligned with the BioEdit program using the ClustalW alignment algorithm. Several conserved gene regions were found in the alignment.

These conserved amino acid sequences were used as a starting point in the design of broad-range primers. First they were reverse-translated to the corresponding nucleic acid sequences. Because of the nature of the genetic code several degenerated sites were observed in these nucleic acid sequences. After this, based on the conserved sequences, various primer pairs were synthesized (ordered from Sigma-Genosys, England, www.sigma-genosvs.co.uk) and tested for specificity and sensitivity. The specificity was tested by amplifying DNA isolated from bacterial species presented in Table 2 using the method described below in Example 4. The primers that amplified rpoB genes of all studied bacterial species were selected as broad-range PCR primers, i.e. primers rpoB2-for which contains the sequences GCYGGNCGHCAYGGWAAYAARGG (SEQ. ID. NR. 20), and RPOb2-rew, which sequence is GGYACSCCVAGDGGGTTYA (SEQ. ID. NR 21), wherein D represents base A or G or T, Y represents base C or T, N represents base A or G or C or T, H represents base A or C or T, W represents base A or T, R represents base A or G, S represents base C or G and V represents base A or C or G (cf. Table 1).

The conserved sequences of the rpoB genes of all bacterial species presented in Table 2 were identified by this mixture of primers and it also amplifies DNA from clinical samples (see Example 6). In particular, this mixture of primers can be used to amplify the rpoB genes of bacteria (Table 2) that are phylogenetically far from each other even in a situation where the sample includes large amounts of human DNA.

TABLE 2
Bacterial strains used in testing of rpoB
PCR primers and oligonucleotide probes.

| Bacterial species | Supplier's code (type of sample) |
|---|---|
| Moraxella catarrhalis | DSM 9143 (bacterial species) |
| Moraxella cuniculi | ATCC VR 1355(bacterial species) |
| Moraxella caviae | ATCC 14659 (bacterial species) |
| Neisseria gonorrhoeaea | ATCC 53420D (DNA) |
| Haemophilus influenzae | ATCC 51907D (DNA) |
| Haemophilus ducreyi | DSM 8925 (bacterial species) |
| Haemophilus parainfluenzae | DSM 8978 (bacterial species) |
| Streptococcus pyogenes | DSM 20565 (bacterial species) |
| Streptococcus pneumoniae | DSM 20566 (bacterial species) |
| Streptococcus oralis | DSM 20627 (bacterial species) |
| Streptococcus mitis | DSM 12643 (bacterial species) |
| Fusobacterium necrophorum | DSM 20698 (bacterial species) |
| Pseudomonas aeruginosa | DSM 50071 (bacterial species) |
| Corynebacterium diphtheriae | DSM 44123 (bacterial species) |
| Legionella pneumophila | ATCC 33152D (DNA) |
| Escherichia coli | DSM 30083 (bacterial species) |
| Pasteurella pneumotropica | ATCC 13669 (bacterial species) |
| Staphylococcus aureus | DSM 20231 (bacterial species) |
| Mycoplasma pneumoniae | ATCC 51907D (DNA) |

ATCC = American Type Culture Collection
DSM = Deutsche Sammlung von Mikroorganismen und Zellkulturen

### EXAMPLE 2

### Production of New Sequences required in the Design of Oligonucleotide Probes as defined by the Invention by Cloning

rpoB sequences of the bacterial species Moraxella catarrhalis, Moraxella cuniculi, Moraxella caviae, Neisseria gonorrhoeae, Haemophilus ducreyi, Haemophilus parainfluenzae, Streptococcus oralis, Streptococcus mitis, Cotynebacterium diphtheriae, Legionella pneumophila ja Pasteurella pneumotropica were sequenced according to the general method described below in order to design oligonucleotide probes of the invention.

First DNA is isolated from bacterial culture isolates using the QlAamp DNA Mini kit (Qiagen, Germany). When DNA has been isolated, the desired target strand used for cloning is amplified using symmetric (conventional) polymerase chain reaction (PCR). In the first step of amplification, the reaction mixture is prepared by mixing the DNA isolated from the sample, the broad-range bacterial primers (Example 1), and the other components needed in the amplification step.

Thus, the reaction mixture (25 μl) used in cloning PCR contains 20 pmol of primer mixture rpoB2-for, 20 pmol of primer mixture RPOB2-rew, 200 μM of each of dATP, dGTP, dTTP, and dCTP (Sigma, USA), 1×Hot Start Taq PCR buffer (Qiagen, Germany), which includes MgCl₂ in order to achieve a final concentration of 2.8 mM, 1.25 U Hot Start Taq DNA polymerase (Qiagen, Germany), and 2.5 μl of isolated DNA.

The cloning PCR is carried out in a GenAmp PCR system 2700 thermal cycler (Applied Biosystems) using the following program: a 15 min denaturation step at 95° C., 38 cycles of 35 s at 94° C., 40 s at 54° C., 35 s at 72° C., and finally a 7 min extension step at 72° C. After the PCR has been performed, the success of amplification is verified by gel electrophoresis using a 2% agarose gel containing ethidium bromide.

Cloning is performed immediately after PCR using a TOPO TA Cloning Kit (Invitrogen, USA). The reaction mixture for cloning contains 4 μl of the PCR product, 1 μl of a salt solution (1.2 M NaCl, 0.06 M MgCl₂), and 1 μl of TOPO vector (pCR 4-TOPO), which are mixed together in an eppendorf tube.

The mixture is incubated for 5 min at room temperature, after which the solution is transferred onto ice. After this chemical transformation is performed, in which 2 μl of cooled cloning mixture is transformed into 50 μl of competent TOPO10 E. coli cells. The transformed cells are incubated for 10 min on ice. In the next stage, a heat-shock treatment is performed. The tube containing the cells is transferred to a 42° C. water bath for 30 s. After this the tube is immediately transferred onto ice and 250 μl of SOC medium at room temperature is added (2% tryptone, 0.5% yeast extract, 10 mM NaCl, 2.5 mM KCl, 10 mM MgCl₂, 10 mM MgSO₄, 20 mM glucose). The tube is shaken horizontally (200 35 rpm) at 37° C. for 1 hour. After this 20 μl of cloning mixture is spread on a prewarmed selective LB plate (Luria-Bertani, 10% tryptone, 0.5% yeast extract, 1.0% NaCl, 1.5% L-agar diluted in water, pH 7), which contains 50 g/ml of ampicillin. The plate is incubated overnight at 37° C. On the next day, ten colonies are chosen from the plate and sequencing PCR is performed.

The reaction mixture (50 μl) for sequencing PCR contains 0.4 pmol of M13 reverse (5′-CAGGAAACAGCTATGAC-3′) and M13 forward (5′-GTAAACGACGGCCAG) primers (provided by the kit), 150 μM of each of dATP, dGTP, dTTP, and dCTP (Sigma, USA), 1×Hot Start Taq PCR buffer (Qiagen, Germany), 1 U Hot Start Taq DNA polymerase (Qiagen, Germany).

For the amplification reaction, a small part of bacterial colony is transferred with the help of a sample stick to the PCR reaction tube.

The sequencing PCR is carried out in a GenAmp PCR system 2700 thermal cycler (Applied Biosystems) using the following program: a 15 min denaturation step at 95° C., 30 cycles of 1 min at 94° C., 1 min at 55° C., 1 min at 72° C., and finally a 10 min extension step at 72° C. After the PCR has been performed, the success of amplification is verified by gel electrophoresis using a 2% agarose gel that contains ethidium bromide. After this, the PCR product is purified by removing additional primers, nucleotides, buffer and polymerase enzyme with a QlAquick PCR purification kit (Qiagen, Germany).

After the purification step the fragment inserted into the vector is sequenced. A 12 μl reaction mixture for the sequencing step contains 100 ng PCR product and 5 pmol of either M13 reverse or M13 forward primer. The sequencing is carried out by a BigDye Terminator Version 3.0 kit and an ABI-PRISM 3100 Genetic Analyzer (Applied Biosystems, USA). The sequences are analyzed with the Vector NTI Suite Version 7 program (InforMax, USA).

With the above-described general method rpoB sequences were produced for the following bacterial species: Moraxella catarrhalis, Moraxella cuniculi, Moraxella caviae, Neisseria gonorrhoeae, Haemophilus ducreyi, Haemophilus parainfluenzae, Streptococcus oralis, Streptococcus mitis, Corynebacterium diphtheriae, Legionella pneumophila and Pasteurella pneumotropica (SEQ. ID: NR: 22 to 32).

### EXAMPLE 3

### Design of Species-Specific Probes

In the design of bacterial species-specific oligonucleotides, i.e. probes, a planning strategy based on alignment was used. The rpoB genes of target bacterial species were aligned with correspondent genes of a few reference bacteria (closely related bacterial species). For example, the rpoB gene of Streptococcus pneumoniae was aligned with rpoB genes of Streptococcus pyogenes, Streptococcus mitis, Streptococcus oralis, Staphylococcus aureus, and Fusobacterium necrophorum. S. oralis and S. mitis are closely related to S. pneumoniae. Therefore, oligonucleotides designed for S. pneumoniae must not react with these normal flora bacteria. Sequences were obtained from the EMBL public sequence database or they were produced by cloning as described in Example 2.

Alignment of the sequences was performed with the BioEdit program using the ClustalW alignment algorithm. The consensus sequence of the alignments was calculated and the suitably conserved regions were identified manually. These regions refer to sequence fragments that are conserved in the genes of the target bacterial species and that are not found at least entirely from the genes of the reference bacteria. Oligonucleotide sequences with the suitable length (19-26 nucleotides) were selected from these areas for comparison analyses. The selected oligonucleotide sequences were compared to the EMBL prokaryotic sequence database using the FastA algorithm program. The oligonucleotide sequences having at least two mismatches when compared to rpoB sequences of non-target bacteria were chosen for further analyses. Theoretical melting temperature (Tm) was determined for oligonucleotides and the formation of secondary structures was studied. Tm (° C.) was calculated with the equation
81.5+16.6 log [Na]+0.41 (% GC)−0.61 (% for) 500/N,

in which Na is the concentration of monovalent cations (50 M is used in calculations), % GC is the proportion of guanine and cytosine, % for is the concentration of formamide (0% is used in calculations) and N is the length of the oligonucleotide. The formation of secondary structures was studied using a program provided by Sigma-Genosys. The program can be used with the help of a web browser at the Internet address http://www.sigma-genosys.co.ukloligos/frameset.html (calculators/basic calculator). The oligonucleotides that did not form strong secondary structures and whose Tm temperature was at least 45° C. were chosen for experimental specificity testing.

Oligonucleotide probes were synthesized and simultaneously modified from the 5′ terminus (NH₂-modified oligos) (Sigma-Genosys, England). The specificity of the probes was tested in the laboratory with DNA samples isolated from different bacterial species (Table 2) and from patient samples (Table 4) as described in Examples 4 and 5 and 6. Of the tested probes, those that functioned best and had the highest specificity were selected. The sequences and specificity of the bacterial species-specific probes are presented in Table 3.

TABLE 3

rpoB oligonucleotide sequences.

| | |
|---|---|
| Oligonucleotide/ | Specificity |

| | | |
|---|---|---|
| SEQ. ID: NR: | Sequence (5′-3′) | (rpoB gene) |

| | | |
|---|---|---|
| 1 | GTTATCTCGAAAATTA | Haemophilus |
| | ACCCAGTTG | influenzae |
| 2 | CGATGAAAATGGTCAG | Haemophilus |
| | CCAGTTGAA | influenzae |
| 3 | GTCGTTTCACGTATTG | Streptococcus |
| | TACCAGT | pyogenes |
| 4 | TTCCAGACGGAACACC | Streptococcus |
| | AGTTGAC | pyogenes |
| 5 | TTCCAGACGGAACTCC | Streptococcus |
| | AGTCGA | pneumoniae |
| 6 | CAGACGGAACTCCAGT | Streptococcus |
| | CGACAT | pneumoniae |
| 7 | CAACGGCACCCCGGTC | Pseudomonas |
| | GACAT | aeruginosa |
| 8 | TGGAAGACATGCCGCA | Pseudomonas |
| | CGAT | aeruginosa |
| 9 | GCCTGTTGAGGATATG | Legionella |
| | CCACA | pneumophila |
| 10 | TGGAAGATGGAACAGC | Legionella |
| | AGTAGACA | pneumophila |
| 11 | TACGATGAAAACGGTA | Escherichia |
| | CTCCG | coli |
| 12 | CAACCCGATCGAAGAT | Escherichia |
| | ATGCC | coli |
| 13 | TATGCCTTACTTACCA | Staphylococcus |
| | GATGGAC | aureus |
| 14 | TACCAGATGGACGTCC | Staphylococcus |
| | GATC | aureus |
| 15 | CAGTAGCGGACATGCC | Mycoplasma |
| | CCA | pneumoniae |
| 16 | TTAGAAGATGGTACTC | Mycoplasma |
| | CAGTCGACA | pneumoniae |
| 17 | ATGGCGGACGGCCGTC | Neisseria |
| | CTGTG | gonorrhoeae |
| 18 | AAATGGTAATCCTGTA | Moraxella |
| | GATATCGTAC | catarrhalis |
| 19 | CTGCCTCAGGAAGATA | Corynebacterium |
| | TGCCAT | diphtheriae |

### EXAMPLE 4

### Amplification of DNA Isolated from Patient Samples

DNAs isolated from bacterial culture isolates or clinical patient samples were amplified and labeled using the conventional method described below.

DNA is isolated from the sample to be analyzed (a bacterial culture or a clinical patient sample) using the QlAamp DNA Mini kit (Qiagen, Germany). When DNA has been isolated, the desired target strand is amplified using asymmetric polymerase chain reaction (PCR). In the first stage of the amplification, a reaction solution is prepared by mixing together DNA isolated from samples, broad-range rpoB2-for and RPOb2-rew primer mixtures (Example 1), and other components needed in the amplification.

The PCR reaction mixture contains 32 pmol of RPOb2-rew primer mixture, 8 pmol of rpoB2-for primer mixture, 200 μM of each of dATP, dGTP, and dTTP as well as 140 μM dCTP (Sigma, USA), 1×Hot Start Taq PCR buffer (Qiagen, Germany), in which MgCl₂ has been added so that a final concentration is 2.8 mM, 2.5 nmol Cy5-AP3-dCTP (Amersham Pharmacia Biotech, USA), 1.25 U Hot Start Taq DNA polymerase (Qiagen, Germany), and 2.5 μl isolated DNA in a total volume of 25 μl.

The PCR is performed using the GenAmp PCR system 2700 thermal cycler (Applied Biosystems, USA). The following PCR program was used: a 15 min denaturation step at 95° C., 38 cycles of 35 s at 94° C., 40 s at 54° C., 35 s at 72° C., and finally a 7 min extension step at 72° C. After the PCR has been performed, the success of amplification is verified by gel electrophoresis using a 2% agarose gel that contains ethidium bromide. After this the Cy5-labeled PCR product is purified by removing additional primers, nucleotides, buffer, and polymerase enzyme with a QlAquick PCR purification kit (Qiagen, Germany).

### EXAMPLE 5

### The Design and Functioning of Sample Microarrays

Oligonucleotide probes aminated at the 5′ terminus (designed according to Example 3) were dissolved in 400 mM sodium carbonate buffer (pH 9.0) to a final concentration of 50 μM. The probes were covalently attached onto aminosilane coated microscope slides (Genorama, Asper Biotech Ltd., Estonia). The transfer of the probes to the glass slides was performed with a robot developed for this purpose (OmniGrid, GeneMachines, USA) and pins (Telechem SMP3, USA). The average size of the printed probe area was 120 μm. Moreover, positive control primers aminated at the 5′ terminus were printed onto the glass slides. After printing the microarray slides were kept in ammonia vapor for 1 hour in order to attach the probes to the slides. After the ammonia treatment they were washed three times with sterile water and dried.

Next, a Cy5-labeled target strand (manufactured according to Example 4) was hybridized to the microscope slide where the probes had been attached. The hybridization reaction mixture contained about 200-300 ng target strand, 20×SSC (1 μl 20×SSC contains 175.3 g NaCl and 88.2 g sodium citrate, pH is adjusted to 7.0 with HCl; the final concentration is 3.4×), 2 μl of 10% sodium dodecyl sulphate (SDS) (a final concentration of 0.3%), and sterile water so that the volume of the reaction mixture was 37 μl. First the mixture was denatured at 95° C. for 3 min. After this the tubes were immediately transferred onto ice. After the mixture had cooled down, it was pipetted onto the cover slip that was placed against the glass slide on which the probes had been attached. The microarray slide was placed inside the hybridization chamber (Arraylt, TeleChem International, USA) and the chamber was shut tight. Finally, the hybridization chamber was immersed in a water bath. The microarray slides were hybridized at 57° C. for 14-16 hours.

After hybridization the microarray slides were washed in three different washing solutions in order to remove non-hybridized DNA. The washing steps were carried out as follows: in 0.1% SDS solution for 5 min at 57° C., in 0.1% SDS, 0.5×SSC washing solution for 5 min at room temperature, and in 0.06×SSC for 5 min at room temperature.

After the glass slides had dried, they were analyzed with a microarray scanner (Agilent DNA Microarray Scanner, Agilent, USA). If the Cy5-labeled target strand had bound to one or several probes, these spots emitted a fluorescent signal. Furthermore, positive control probes also gave a fluorescent signal.

An example of a hybridization result is presented in FIG. 3. A rpoB DNA fragment (an asymmetric Cy5-labeled PCR product) isolated from a culture isolate of Streptococcus pneumoniae (pathogen) and Streptococcus oralis (normal flora) belonging to the same species was used as the target strand. On the example slide the arrow marks the oligonucleotide spots binding the labeled target strand of S. pneumoniae. The oligonucleotide sequences on the slide are S. pneumoniae oligonucleotide probes 5 and 6 as shown in Table 3. Also the oligonucleotide spots containing the positive control oligonucleotide (broad-range PCR primer SEQ. ID. NR. 21) gave a signal on both slides. The positive controls spots are markers for a successful hybridization, although no bacterial-specific binding is detectable. S. oralis-specific oligonucleotide spots are not detected on the glass slide. The comparison between the two figures proves that the S. oralis (normal flora) does not cross react with S. pneumoniae (pathogen) oligonucleotide spots. This is because on the other microarray slide that was hybridized with the amplified rpoB DNA fragment of S. oralis no bacterial-specific hybridization has taken place. The S. pneumoniae target strand did not hybridize to any other oligonucleotide spots on the slide.

### EXAMPLE 6

### Analysis of Patient Samples

DNA from clinical samples obtained from patients suffering from respiratory tract infections was isolated and amplified using the method described in Example 4. The samples were tested using the microarray slide (described in Example 5) on which the probes and positive control oligonucleotides (listed in Table 3) were attached. The same samples were also analyzed with the culture testing. The summary of the results is shown on Table 4. The results obtained with the method of the invention are entirely identical when compared to the culture testing of prior art. The method according to the present invention is substantially faster to perform than the culture testing, as when performing the culture testing the results are available in approximately one day.

TABLE 4
Comparison between the method of the present
invention and the culture testing

| Otitis media, | Culture | Hybridization |
|---|---|---|
| suppuration sample | result | result |
| C130 | Sp, Hi | Sp Hi |
| C131 | Sp | Sp, Sa |
| C132 | Sp, Sa | Sp, Sa |
| C156 | Hi, Cd | Hi, Cd |
| C146 | Hi | Hi |

The abbreviations used in the Table:
Sp—Streptococcus pneumoniae, Hi—Haemophilus influenzae, Sa—Staphylococcus aureus, and Cd—Corynebacterium diphteriae.

1. A diagnostic method for detecting and identifying bacterial species causing infections from a clinical sample, said method comprising:
a) amplifying DNA isolated from said clinical sample using a mixture of DNA primers that comprises sequences which hybridize with the sequences that originate from conserved regions of rpoB genes encoding DNA directed RNA polymerase [EC:2.7.7.6] subunit B of bacterial species causing infections, said sequences comprising SEQ ID NOS: 20 and 21 and/or complementary sequences thereof and/or functional fragments thereof,
b) contacting the amplified DNA with a desired combination of oligonucleotide probe sequences that hybridize under normal hybridization conditions with hyper-variable regions situated near said conserved regions of rpoB genes encoding DNA directed RNA polymerase [EC:2.7.7.6] subunit B of bacterial species causing said infections, said sequences being bacterial species specific under said hybridization conditions, and
c) detecting the formation of a possible hybridization complex.

2. The diagnostic method according to claim 1, wherein said infections causing bacterial species are bacterial species that cause human disease, particularly respiratory tract infections and/or ear, nose and throat diseases.

3. The diagnostic method according to claim 1, wherein said hyper-variable region is the hyper-variable region of the gene encoding the rpoB protein of a bacterial species selected from Haemophilus influenzae, Streptococcus pneumoniae, Streptococcus pyogenes, Pseudomonas aeruginosa, Staphylococcus aureus, Legionella pneumophila, Corynebacterium diphteriae, Mycoplasma pneumoniae, Escherichia coli Moraxella catarrhalis and Neisseria gonorrhoeae.

4. The diagnostic method according to claim 1, wherein the length of oligonucleotide probe sequences used in step b) 15-30, more preferably 19-30, and most preferably 19-26 nucleic acids and are optionally labeled.

5. The diagnostic method according to claim 1, wherein said combination of oligonucleotide probe sequences comprises all or a portion of SEQ ID NOS: 1 to 19, and/or complementary sequences thereof, or functional fragments thereof and preferably it comprises all of the SEQ ID NOS: 1 to 19.

6. The diagnostic method according to claim 5, wherein said combination of oligonucleotide probe sequences is attached onto a solid support, preferably onto treated glass.

7. The diagnostic method according to claim 1, wherein the DNA isolated from the clinical sample in step a) is amplified using the polymerase chain reaction (PCR) and wherein the DNA amplified in step b) is contacted with the bacterial species-specific oligonucleotide probes attached onto a solid support.

8. The diagnostic method according to claim 7, wherein suitably labeled nucleotides are used in the amplification of DNA isolated from a clinical sample in step a) to generate a detectable target strand and wherein the amplified and optionally labeled target DNA in step b) is contacted with a solid support, on which all bacterial species-specific oligonucleotide probes of SEQ ID NOS: 1 to 19 and/or complementary sequences thereof have been attached.

9. The diagnostic method according to claim 8, wherein the amplified and optionally labeled target DNA in step b) is contacted with a solid support, preferably treated glass, on which specific oligonucleotide probe sequences detecting one specified bacterial species or a few specified bacterial species causing infections have been attached, said sequences being selected from sequences shown in Table 3 and/or complementary sequences thereof.

10. The diagnostic method according to claim 1, wherein the microarray technology is used in step c).

11. A DNA primer mixture comprising sequences that hybridize with sequences of the conserved regions of rpoB genes encoding DNA directed RNA polymerase [EC:2.7.7.6] subunit B of bacterial species that cause infections, said mixture comprising SEQ ID NOS: 20 and 21 and/or complementary sequences thereof or functional fragments thereof.

12. An oligonucleotide sequence useful in the diagnosis of infection causing bacterial species, wherein said oligonucleotide sequence hybridizes under normal hybridization conditions with a sequence of a hyper-variable region that is bacterial species-specific and is situated near the conserved regions of rpoB genes encoding DNA directed RNA polymerase [EC:2.7.7.6] subunit B of bacterial species causing said infections, said oligonucleotide sequence being bacterial species-specific and said oligonucleotide sequence comprising one of the SEQ ID NOS: 1 to 19 or complementary sequences thereof or functional fragments thereof.

13. The combination of oligonucleotide probe sequences useful in the diagnosis of infection causing bacterial species comprising any combination of the SEQ ID NOS: 1 to 19 or complementary sequences thereof or functional fragments thereof.

14. The combination of oligonucleotide probes according to claim 13 comprising all of the SEQ ID NOS: 1 to 19.

15. The use of the combination of oligonucleotide probes according to claim 14 for the detection, identification, or classification of disease causing bacterial species.

16. A diagnostic kit for use in the diagnosis of infection-causing bacteria, especially those causing respiratory tract infections, comprising
a) a DNA primer mixture comprising sequences that hybridize with sequences of the conserved regions of rpoB genes encoding DNA directed RNA polymerase [EC:2.7.7.6] subunit B of bacterial species causing infections, especially bacterial species that cause respiratory tract infections, said mixture comprising SEQ ID NOS: 20 and 21 or complementary sequences thereof or functional fragments thereof,
b) a combination of bacterial species-specific oligonucleotide probe sequences, optionally attached on a solid support, comprising any combination of the SEQ ID NOS: 1 to 19 or complementary sequences thereof or functional fragments thereof,
c) positive and optionally negative control probe sequences, and optionally
d) reagents required in the amplification, hybridization, purification, washing, and/or detection steps.
